(19) United States
(12) Patent Application Publication
(10) Pub. No.: US 2006/0200168 A1
(43) Pub. Date: Sep. 7, 2006

(54) System and method for providing access in divergent directions in a vascular environment

(76) Inventors: Azam Anwar, Dallas, TX (US); Georges A. Feghali, Dallas, TX (US)

Correspondence Address: BAKER BOTTS L.L.P., 2001 ROSS AVENUE, SUITE 600, DALLAS, TX, 75201-2980, US

(21) Appl. No.: 11/071,006

(22) Filed: Mar. 3, 2005

(51) Int. Cl. A61F 11/00

(52) U.S. Cl. 606/108; 606/191; 600/585

(57) ABSTRACT

A system for providing optimal access in a vascular environment is provided that includes a first lumen operable to be used in a procedure involving a vessel of a patient and a second lumen operable to be inserted in conjunction with the first lumen in the procedure. A first wire is provided and inserted in the first lumen and positioned in a first direction. A second wire is provided and used in the second lumen and positioned in an opposite direction, which is not the same as the first direction. In a more particular embodiment, the above-identified system may include a dilator operable to dilate the vessel in conjunction with the procedure and a sheath operable to deliver the first and second lumens for the procedure.

### TECHNICAL FIELD OF THE INVENTION

This invention relates in general to vascular procedures and, more particularly, to a process, a system, and a method for providing access in divergent directions in a vascular environment.

### BACKGROUND OF THE INVENTION

The treatment of vascular diseases has grown exponentially in terms of sophistication and diversity. Procedures involving items such as stents and balloons are virtually routine in many health-care practices. One issue associated with any vascular procedure relates to how to provide for optimal access to multiple anatomical sites. This is particularly problematic in scenarios where several operations, which are performed during a substantially equivalent timeframe, require access to two different areas of the patient.

For example, once a tube (e.g. a catheter or a sheath) is introduced into a given vessel of the patient, that same tube (or a wire introduced through that tube) may need to be redirected such that it points in another direction. Hence, the physician may be required to perform two separate tasks for that patient during roughly the same time period. In many cases, the subsequent procedure must be postponed such that the same side of the patient can be accessed at a later time. If the physician insists on carrying out the second procedure immediately after finishing the first procedure, then a second incision is often necessary. Thus, a patient must generally be repunctured, whereby a second tube is inserted into the patient and subsequent work is performed in the targeted area.

This is not an ideal scenario because there is already an existing instrument in the patient. Moreover, making a second puncture in the patient is time-consuming and may further involve guiding an instrument of choice from one puncture site to the targeted region (e.g. probing from one leg over to the opposite leg where the procedure was initiated). Because of this increased difficulty, a physician may opt not to perform this additional procedure.

In other cases, which may involve antegrade wire applications, a physician may attempt to strategically puncture the patient (e.g. incise at a higher anatomical location) in order to complete some of these operations. However, such a protocol may not be safe, as there are a number of salient hazards with this approach (e.g. the procedure may be difficult to perform if the patient is obese). Accordingly, the ability to properly accommodate multi-directional access during a given vascular procedure presents a significant challenge for physicians.

### SUMMARY OF THE INVENTION

From the foregoing, it may be appreciated by those skilled in the art that a need has arisen for an improved process for achieving superior multi-directional access during any number of vascular procedures. In accordance with an embodiment of the present invention, a device, a system, and a method for facilitating access to multiple patient sites are provided that substantially eliminate or greatly reduce disadvantages and problems associated with conventional vascular disease approaches, strategies, and instruments.

According to an embodiment of the present invention, a system for offering divergent access is provided that includes a first lumen operable to be used in a procedure involving a vessel of a patient and a second lumen operable to be used in conjunction with the first lumen in the procedure. A first wire is provided and inserted into the first lumen and positioned in a first direction. A second wire is provided and inserted into the second lumen and positioned in an opposite direction, which is not the same as the first direction. In a more particular embodiment, the above-identified system may include a dilator operable to dilate the vessel in conjunction with the procedure and the same dilator is operable to deliver the first and second lumens for the procedure.

Certain embodiments of the present invention may provide a number of technical advantages. For example, according to one embodiment of the present invention, an architecture and a process are provided that offer a flexible system, which can easily accommodate simultaneous access to several regions of the patient. This would provide a capability for the physician to perform multiple tasks during a single procedure: both in upstream and downstream directional flows. This, in turn, would allow the present invention to have a broad range of applications, and, thus, be used in any number of (potentially challenging) environments. Note that in many instances, the physician may be standing awkwardly: potentially in a strenuous position (e.g. to obtain antegrade access). In addition, the physician may be required to perform many vascular-type tasks with his left hand (where the physician is positioned on the right side of the patient and is generally right-handed). The present invention offers an optimal solution, which ameliorates these strains and reduces the burden placed on the tending physician.

Moreover, the proffered system of the present invention offers some degree of familiarity for practicing physicians. This is due to the fact that most physicians, who work in this area, have experience in the use of retrograde wires, as these procedures are somewhat routine in most vascular practices. Thus, introducing a retrograde wire in order to later introduce an antegrade wire presents a protocol that should achieve some level of comfort for even the most novice physicians.

Certain embodiments of the present invention may enjoy some, all, or none of these advantages. Other technical advantages may be readily apparent to one skilled in the art from the following figures, description, and claims.

### BRIEF DESCRIPTION OF THE DRAWINGS

To provide a more complete understanding of the present invention and features and advantages thereof, reference is made to the following description, taken in conjunction with the accompanying figures, wherein like reference numerals represent like parts, in which:

FIG. 1 is a simplified schematic diagram illustrating a patient that may subjected to an example vascular procedure;

FIG. 2A is a simplified schematic diagram of a device that may be used on the patient of FIG. 1;

FIG. 2B is a simplified schematic diagram of the device from a top view perspective;

FIG. 3 is a simplified schematic diagram of an example operation of the system;

FIG. 4 is a simplified schematic diagram of a resultant, which reflects a subsequent step completed in the example operation of FIG. 3;

FIGS. 5A-C are simplified schematic diagrams of various example configurations of the system during a portion of a procedure that involves some of the teachings of the present invention; and

FIGS. 6A-F are a series of simplified schematic diagrams, which illustrate an example chronology associated with the present invention.

### DETAILED DESCRIPTION OF THE INVENTION

For purposes of teaching and discussion, it is useful to provide some overview as to the way in which the following invention operates. The following foundational information may be viewed as a basis from which the present invention may be properly explained. Such information is offered earnestly for purposes of explanation only and, accordingly, should not be construed in any way to limit the broad scope of the present invention and its potential applications.

Divergent access within a vessel may be appropriate in certain instances. Consider an example in which a catheter or a sheath is inserted in the femoral artery going toward the heart (i.e. against the natural direction of the bloodstream). In certain instances, that same sheath may need to be redirected such that it extends down the leg in the same direction of the bloodstream. For a physician to do this, a second incision is generally necessary in the contralateral femoral artery. Thus, a patient must be repunctured, whereby a second tube is inserted into the femoral artery and then a long tube is directed over a wire from the contralateral femoral artery toward the ipsilateral femoral artery.

Repuncturing is an imperfect solution because there is already an existing sheath in the patient that stretches in an upstream direction. During such a scenario, the (already placed) sheath must be removed or realigned in order to be positioned in this new direction. Hence, this is not an ideal situation because it carries an additional risk, as the patient will be exposed to another puncture and yet another tube will be placed in the patient to accommodate the new procedure. A physician, who is relegated the arduous task of performing work in two directions of the vessel in this case, may opt not to perform this (second) part of the operation during the same setting. If the physician is forced to complete his procedural objective during the same procedure, he would be required to repuncture the patient (for example at roughly the same location, but on the other leg) and then guide an instrument to the targeted region (i.e. channel the instrument from one leg over to the opposite leg where the procedure was initiated). Again, this technique brings additional risks, such as injuring the leg arteries.

The present invention overcomes these problems (and others) in providing divergent access for a tending physician in such scenarios. Consider a routine procedure in which the objective of a physician is to: 1) take a series of pictures; and 2) perform some work on the patient in a downstream direction. A first wire could be introduced into the patient in a traditional manner: opposite to the bloodflow. Subsequently, another wire can be directed through the same puncture. The second wire can then be directed in an opposite direction and used to advance the sheath in that direction such that work can be done in the downstream direction of the blood flow.

Even though there is work being done in (potentially) two directions (or more), the present invention provides a protocol where there is only one physician manipulation. A sole physician has enough dexterity to adequately perform multiple tasks using such an architecture. Additionally, the work can be performed during the same procedure, which avoids the two-puncture scenario discussed above.

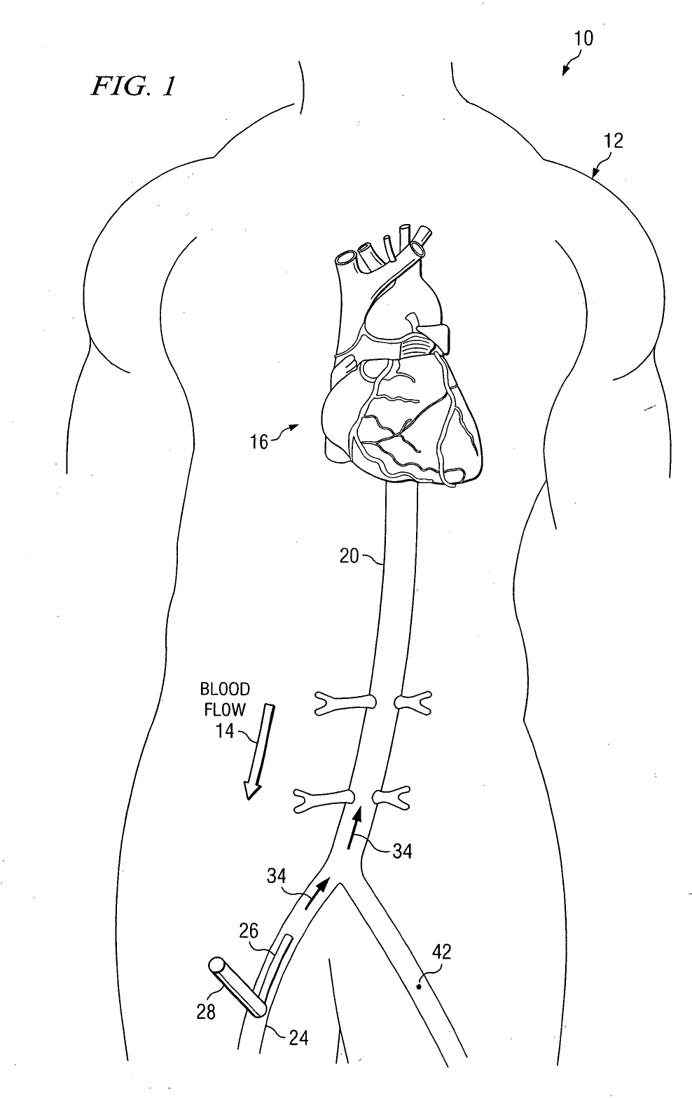

Referring back to FIG. 1, FIG. 1 offers a system 10 that is designed to address the deficiencies highlighted above in providing a divergent access capability for a physician engaged in a given procedure. It is critical to note that, as used herein in this document, the term “divergent” encompasses at least two directions, but is certainly inclusive of multiple directions of access. For example, although procedures outlined herein involve work being performed in the downstream or upstream directions, any variations of these paths may certainly be accommodated by the present invention. Some operations may simply be more lateral, or angled such that they can accomplish a task in a desired location: such operations being not necessarily in an absolute upstream or downstream direction. The present invention is replete with any such alternatives and, therefore, should be interpreted as such.

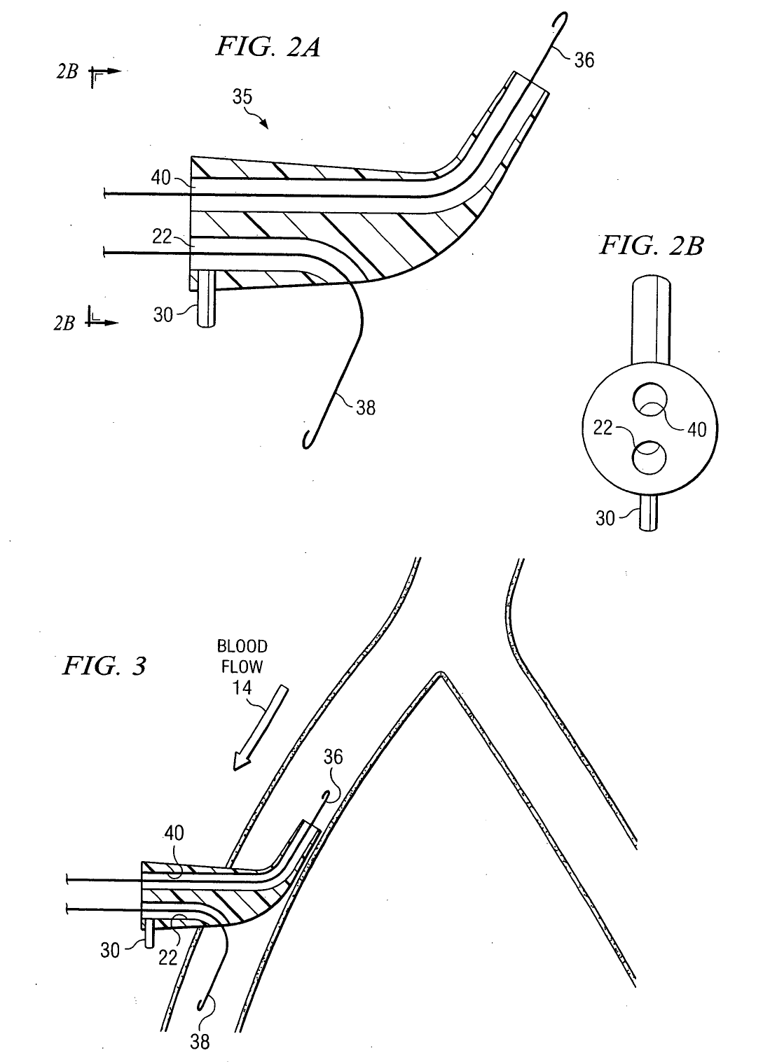

FIG. 1 includes a patient 12, having a heart, which is generally indicated at 16, and an aorta 20 that extends from heart 16, along the spinal cord, and to the legs of patient 12. Note that the introducer device to be employed in system 10 is provided in FIG. 2A for purposes of clarity for the audience. This was done in an effort to further detail the device itself, as it is relatively small in comparison to the area illustrated in FIG. 1.

An arrow 14 is provided in FIG. 1 in order to illustrate the natural direction of blood flow for patient 12. For purposes of performing a typical vascular procedure, a physician may create an entry point 24, from which a catheter 26 may be inserted. A simple gripping mechanism or an introductory element 28 (e.g. a plastic tube) may be provided to adjust or to manipulate catheter 26 to any suitable location. Introductory element 28 is generally just part of the introductory sheath system, which can accommodate particular curvatures and remain at entry point 24 of patient 12 throughout the procedure. In certain embodiments, introductory element 28 can be used for flushing operations and, further, be used to ensure that air or clots are not present during the procedure. The introductory sheath is generally sufficient to accommodate all of the equipment identified herein (e.g. balloon, stent, lumen, etc.).

Catheter 26 may be fed through entry point 24 and propagate along aorta 20 until it reaches its intended destination. A series of arrows 34 are provided in order to illustrate one example pathway associated with a first procedure of system 10, whereby the attending physician may guide catheter 26 in any suitable fashion. In operation of an example embodiment, a sheath may be placed in the right or left groin, whereby entry point 24 is incised at the femoral artery. The sheath would be positioned at entry point 24. The sheath is simply a tube of some type (e.g. plastic, glass, etc.) that can be used as a conduit and from which other instruments may be delivered.

Note that a second entry point 42 is also illustrated in FIG. 1. Second entry point 42 is undesirable, as it reflects some of the problems outlined above. Without use of the present invention, entry point 42 would need to be created and utilized to accommodate subsequent procedures, which require work being performed in a different bloodflow direction.

Turning to FIG. 2A, FIG. 2A is a simplified schematic of an introducer device 35 that may be used within system 10 of FIG. 1 to achieve the teachings of the present invention. FIG. 2B simply offers a top perspective of introducer device 35. FIG. 2A includes a first lumen 40 (also referred to as lumen #1 herein) and a second lumen 22 (also referred to an lumen #2 herein). Lumen #1 includes a first wire 36 (also referred to as wire #1 herein) and lumen #2 includes a wire second wire 38 (also referred to as wire #2 herein). Attached to lumen #2 is a port 30, which can be used to offer a bleed off or an aspiration point.

These materials could be included in a given kit, which could ostensibly be provided to a physician who is responsible for performing a divergent procedure. The kit could include: two wires, a standard dilator, a sheath, and the two-lumen introducer device. Any of these components may be manufactured based on particular specifications or specific patient needs. The present invention contemplates considerable flexibility in such components, as any permutation or modification to any of these elements is clearly within the broad scope of the present invention.

It should be noted that as used herein in this document, the term “introducer” connotes a two-lumen device of the present invention, which is capable of receiving two wires. The introducer may be constructed of plastic, metal, glass, or any composite material, which is operable to facilitate the operations of the present invention. Considerable flexibility is provided by the present invention, as virtually any material may be used to form the introducer. In one embodiment, the introducer may be designed such that it has a particular curvature that is generally parallel with the vessel. Alternatively, the introducer may be designed based on particular needs and specific arrangements. Similarly, the introducer may be of any requisite length, diameter, and/or width. Again, these designations and specifications may be made based on specific needs or particular physician and/or patient arrangements and scenarios.

In operation of an example embodiment, catheter 26 could be placed in entry point 24 and propagate along aorta 20. Introducer device 35 (collectively illustrated in FIG. 2A) could be inserted at entry point 24, as the sheath can easily constrain any such instruments. The device would then subsequently offer multi-access capabilities for a given region within a vessel of patient 12. Additional details relating to this process and the features of the device are provided below with reference to the following FIGURES.

FIG. 3 is a simplified schematic diagram of an example operation of the introducer. This example may begin where a physician inserts sheath 26 (in a traditional manner) in the upstream direction and other instruments may also be guided or channeled through this location.

At some point in this example, the administering physician elects to perform some work in the downstream direction. A wire is advanced through the original sheath, which would propagate through lumen #1 and go upstream. Then the original sheath is removed and the introducer device is advanced. This advancement may be done over the original wire and propagate up the femoral artery. Introducer device 35 is then pulled back such that any blood drip from lumen #2 ceases. As the device is being pulled back, it is outside of the arterial lumen. The device may be reinserted back into patient 12 (or moved forward) to ensure that lumen #2 is back with the vessel lumen.

The second hole of lumen #2 is generally on the opposite side of the vessel. Lumen #2 forces or urges wire #2 to go downstream. As can be appreciated, wires generally follow the path of least resistance. In this example, because of the structure of the device and the direction of lumen #2, the wire is encouraged to point in a downstream direction of patient 12.

As identified above, wire 36 is removed and what is left is wire 38, which is directed opposite to the position of wire 36. Wire 38 is provided for antegrade access and is used to deliver the sheath. The tip of wire 38 is directed downstream. Both wires may include a hook on their tips and be pre-bent to a specified angle or elevation. Additionally, the wires may have a designated diameter (e.g. 0.010 to 0.075 inches) and/or be manufactured or designed based on particular needs and configurations.

The hook, which is illustrated as being part of both wire #1 and wire #2, is designed to access a given region of the patient in a desired direction. In one example embodiment, wire #1 comprises retrograde material, which is designed to perform optimally even though it is going against the flow of blood. Similarly, and in a particular embodiment, wire #2 comprises antegrade material, which is designed to perform optimally as it moves with the flow of blood in the downstream direction.

Wire #2 may be used in a host of applications. For example, wire #2 may be used when an intervention is necessary in a region that is downstream of entry point 24. This could include repairing a given area or dilating stenosis (e.g. placing a balloon, stint, etc.). It should also be noted that because of a patient's shape, getting an antegrade access in the standard way can be difficult. This could include cases where the patient's belly is simply too large to accommodate such a procedure. Thus, the present invention presents a practical solution for these individuals, whose body habit restricts a physician's movements such that puncturing the vessel in the correct location is difficult (and potentially harmful to the patient).

Note also that system 10 is generally safer for patient 12 because of the hazards in trying to strategically position an antegrade wire directly into patient 12 initially. This is due to the fact that a vascular procedure, which does not use the introducer device of the present invention, would require an incision into patient 12 at a higher anatomical location. During such a procedure, a needle would be targeted to a higher location of the body: even though the targeted region for an intervention is somewhat lower anatomically. The higher positioning may be generally done to avoid undesirable injury to the profunda femoris, which is a vessel carrying blood towards the inside of the thigh. Attempting to “finesse” such a procedure (i.e. work around the profunda femoris, as well as numerous other branches that are present in this region) would be far less safe than the proposed method, as detailed herein.

It should also be noted that usage of antegrade wires presents a significant challenge for any practicing physician. Without utilization of the present invention, a physician is forced to introduce and to manipulate antegrade wires initially at the outset of the procedure, which is problematic. In many procedures, the physician is forced to stand in a challenging position. In addition, the physician may be required to complete his work with his left hand (where the physician is positioned on the right side of the patient and is generally right-handed). Also, in cases where the belly is excessively large (such being the case in many vascular procedures), it is difficult for a physician to ascertain a good anatomic landmark. This could, in turn, cause complications in sticking the patient too high or too low. These problems highlight only some of the many potential problems facing a physician who must account for antegrade wires and who is not able to implement the introducer device of the present invention.

Note also that system 10 has the added benefit of providing some architectural familiarity for most physicians. This is due to the fact that most physicians have experience in working with retrograde wires, as procedures involving these elements are somewhat routine amongst this peer group. Thus, introducing a retrograde wire in order to later introduce an antegrade wire presents a protocol that is relatively simple: even for inexperienced physicians.

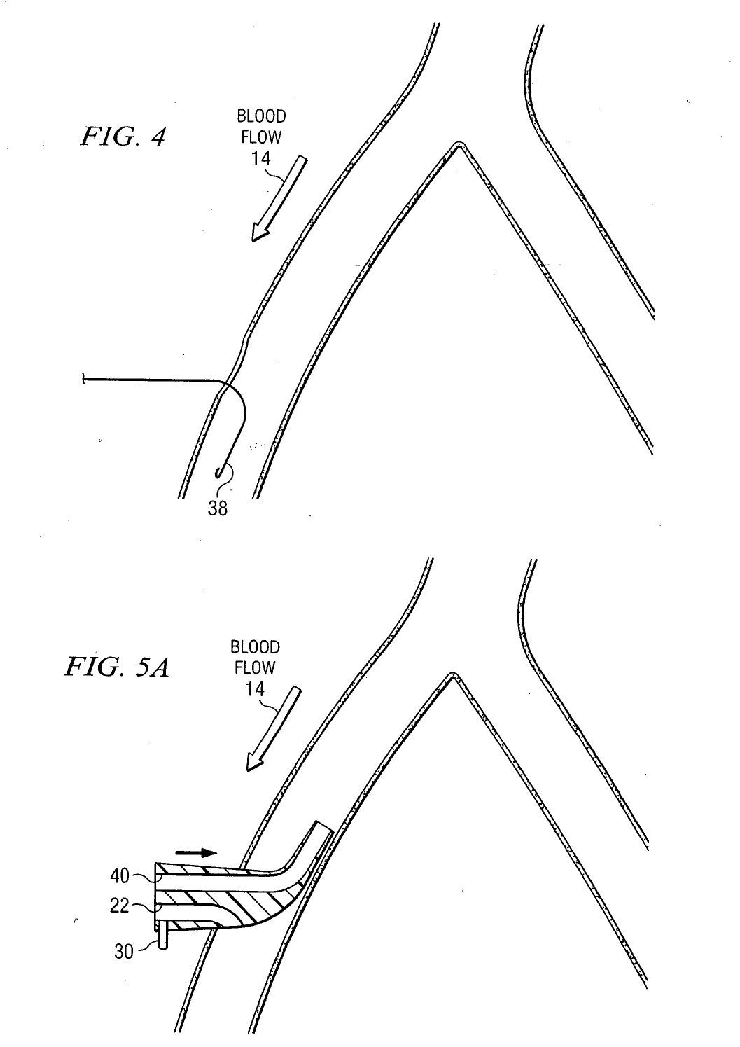

Turning to FIG. 4, FIG. 4 is yet another simplified schematic diagram of an example operation of the introducer device. FIG. 4 illustrates the notion that only one component is left within the aortic region, after the other instruments have been taken out of patient 12. Thus, once wire #2 is properly manipulated such that it is pointing toward the appropriate downward direction, then the majority of the apparatus may be removed. Hence, in one embodiment, once wire #2 is suitably positioned in the downstream path, all other components may be pulled from entry point 24 such that only wire #2 remains. At this point, the sheath may be advanced over wire #2, and ensuing work can then be performed in different directions of the aortic region.

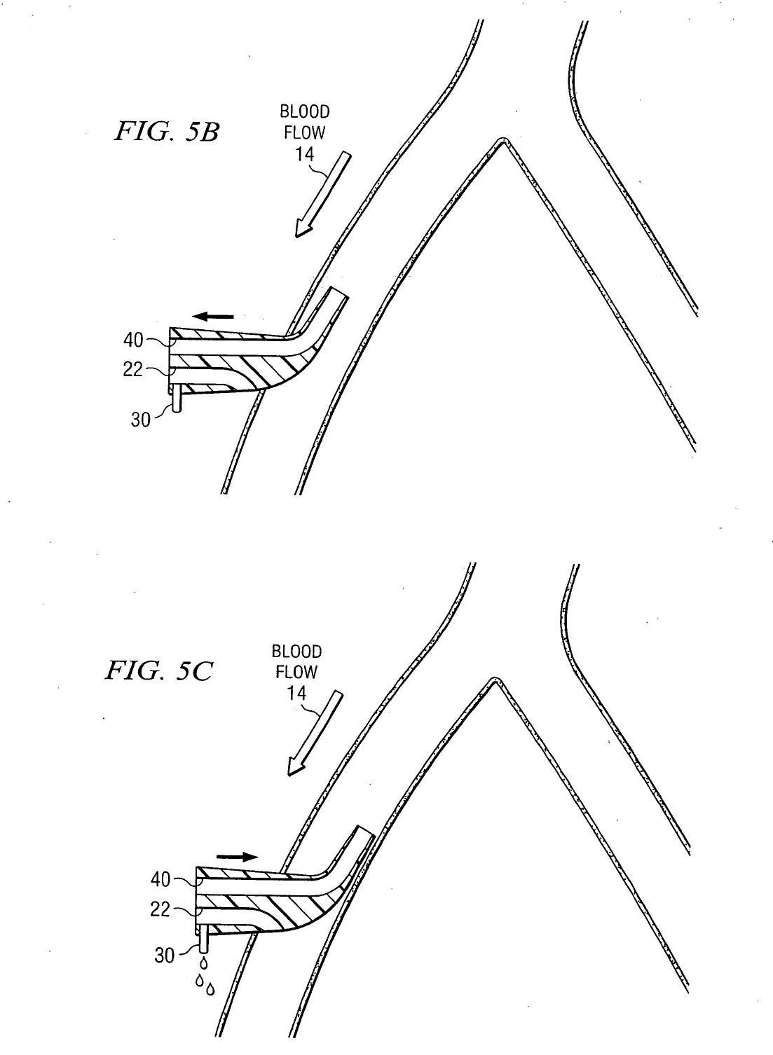

FIGS. 5A-C are simplified schematic diagrams of various example configurations of system 10 during a portion of a procedure that involves the introducer device. FIG. 5A reflects a scenario in which both lumens are within the targeted location. FIG. 5B illustrates a removal of the introducer such that lumen #2 is exterior to the artery. FIG. 5C shows how the introducer can be advanced such that lumen #2 is reengaged within the aortic region.

These FIGURES, collectively, show how a physician would proceed after he has decided to complete some task in the downstream direction. This may be achieved by: 1) inserting the introducer into the patient; 2) pulling back the introducer such that any blood drip from lumen #2 stops; 3) inserting a second wire into the introducer and advancing the introducer such that lumen #2 is within the arterial flow; and 4) removing the introducer such that only the second wire remains. Thus, in the third step, the introducer may be repositioned (or moved forward) to ensure that lumen #2 is back within the vessel, as is clearly shown in FIG. 5C. In one example, the introducer is advanced about 1 centimeter in an attempt to position the second lumen in the middle of the aorta. Other distances may be based on particular configurations. A small amount of blood flow may help to confirm that lumen #2 has been positioned properly (i.e. placed in an adequate intraluminal fashion).

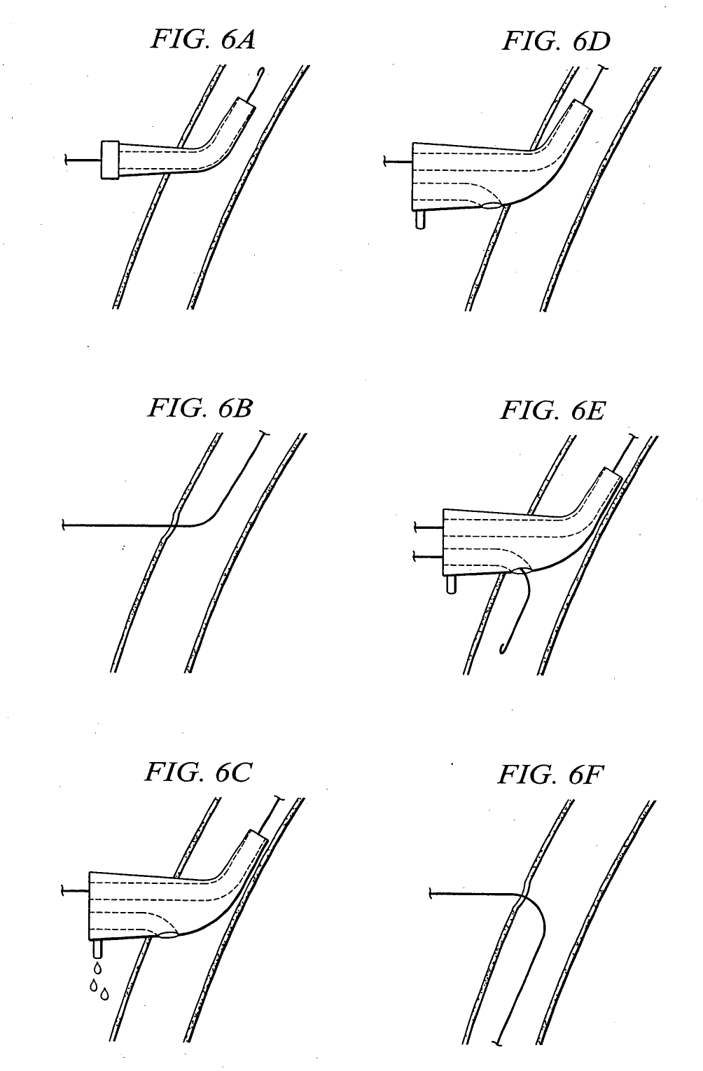

FIGS. 6A-F are a series of simplified schematic diagrams, which illustrate an example chronology associated with the present invention. FIG. 6A illustrates a simple puncture scenario for the patient, whereby a wire is then inserted into the vessel and a sheath is advanced over the wire. FIG. 6B illustrates the sheath being removed such that only the wire remains in the vessel.

FIG. 6C illustrates placement of the introducer over the wire. As is evident by the diagram, both lumens are within the vessel. In FIG. 6D, the introducer is slightly disengaged from the vessel such that the opening of the second lumen is temporarily outside the vessel (the other opening of the first lumen is still within the vessel, which is why one hole is producing a blood drip and the other is not). In FIG. 6E, the introducer is reengaged. In addition, FIG. 6E illustrates that a second wire is inserted into the introducer.

FIG. 6F shows how the first wire and the introducer are removed from the vessel. Also, FIG. 6F illustrates that only the second wire is left in the vessel. The second wire is an antegrade wire, as discussed above. With the second wire in place, any number of subsequent procedures may be performed. For example, the sheath may be advanced over the second wire in order to perform work in the downstream direction of the patient.

It is important to note that the stages and steps in the preceding FIGURES illustrate only some of the possible scenarios that may be executed by, or within, the architecture of the present invention. Some of these stages and/or steps may be deleted or removed where appropriate, or these stages and/or steps may be modified or changed considerably without departing from the scope of the present invention. In addition, a number of these operations have been described as being executed concurrently with, or in parallel to, one or more additional operations. However, the timing of these operations may be altered considerably. The preceding example flows have been offered for purposes of teaching and discussion. Substantial flexibility is provided by the proffered architecture in that any suitable arrangements, chronologies, configurations, and timing mechanisms may be provided without departing from the broad scope of the present invention.

Note also that the example embodiments described above can be replaced with a number of potential alternatives where appropriate. The processes and configurations discussed herein only offer some of the numerous potential applications of the device of the present invention. The elements and operations listed in FIGS. 1-6F may be achieved with use of the present invention in any number of contexts and applications. Accordingly, suitable infrastructure may be included within the system to effectuate the tasks and operations of the elements and activities associated with managing divergent access.

Although the present invention has been described in detail with reference to particular embodiments in FIGS. 1-6F, it should be understood that various other changes, substitutions, and alterations may be made hereto without departing from the sphere and scope of the present invention. For example, although the preceding FIGURES have referenced a number of components as participating in the numerous outlined procedures, any suitable equipment or relevant tools may be readily substituted for such elements and, similarly, benefit from the teachings of the present invention. These may be identified on a case-by-case basis, whereby a certain patient may present a health risk factor while another (with the same condition) may not. Hence, the present device may be designed based on particular needs with specific scenarios envisioned.

It is also imperative to note that although the present invention is illustrated as implicating the femoral artery, this has only been done for purpose of example. The present invention could readily be used in any artery or vessel and, accordingly, should be construed as such. The present invention may easily be used to provide reverse access in arterial and venous procedures at various locations of the mammalian anatomy, which are not necessarily illustrated by FIG. 1.

Numerous other changes, substitutions, variations, alterations, and modifications may be ascertained to one skilled in the art and it is intended that the present invention encompass all such changes, substitutions, variations, alterations, and modifications as falling within the spirit and scope of the appended claims. In order to assist the United States Patent and Trademark Office (USPTO) and additionally any readers of any patent issued on this application in interpreting the claims appended hereto, Applicant wishes to note that the Applicant: (a) does not intend any of the appended claims to invoke paragraph six (6) of 35 U.S.C. section 112 as it exists on the date of filing hereof unless the words “means for” are specifically used in the particular claims; and (b) does not intend by any statement in the specification to limit his invention in any way that is not otherwise reflected in the appended claims.

What is claimed is:

1. An introducer, comprising:
a first lumen operable to be used in a procedure involving a vessel of a patient; and
a second lumen operable to be used in conjunction with the first lumen in the procedure, wherein the first lumen is operable to receive a first wire, which can be inserted in the first lumen and positioned in a first direction, and wherein the second lumen is operable to receive a second wire, which can be inserted in the second lumen and positioned in an opposite direction, which is not the same as the first direction.

2. The apparatus of claim 1, wherein the first lumen and the first wire are used in a first part of the procedure that involves work being performed in an upstream direction with respect to blood flow of the patient.

3. The apparatus of claim 2, wherein the second lumen and the second wire are used in a second part of the procedure that involves work being performed in a downstream direction with respect to the blood flow of the patient.

4. The apparatus of claim 1, wherein the first wire comprises an antegrade material and the first direction is substantially upstream with respect to blood flow of the patient.

5. The apparatus of claim 1, wherein the second wire comprises a retrograde material and the opposite direction is substantially downstream with respect to blood flow of the patient.

6. The apparatus of claim 1, wherein the second lumen includes a port that is operable to perform a bleed off function for the procedure.

7. The apparatus of claim 1, wherein the first and second wires each include a tip that inhibits snagging in the vessel, and wherein the first and second wires are pre-bent to particular specifications.

8. The apparatus of claim 1, further comprising:
a dilator operable to dilate the vessel in conjunction with the procedure.

9. The apparatus of claim 1, wherein a portion of the introducer includes a curvature that is substantially parallel to the vessel.

10. A method, comprising:
inserting a first wire a vessel of a patient;
inserting a sheath over the wire;
inserting an introducer into the vessel, wherein the introducer includes a first lumen and a second lumen;
positioning the introducer; and
inserting a second wire into the second lumen, wherein the first wire is positioned in a first direction of the vessel and the second wire is positioned in an opposite direction, which is not the same as the first direction.

11. The method of claim 10, wherein the first lumen and the first wire are used in a first part of the procedure that involves work being performed in an upstream direction with respect to blood flow of the patient.

12. The method of claim 11, wherein the second lumen and the second wire are used in a second part of the procedure that involves work being performed in a downstream direction with respect to the blood flow of the patient.

13. The method of claim 11, further comprising:
manipulating a port provided in the first lumen in order to perform a bleed off function for the procedure.

14. The method of claim 10, wherein the positioning step further includes:
repositioning the introducer in the patient until intraluminal positioning is achieved, and wherein the intraluminal positioning may be confirmed by a blood leakage from the patient.

15. The method of claim 10, further comprising:
removing the introducer such that only the second wire remains in the vessel, wherein the sheath is operable to be inserted back into the vessel over the second wire such that subsequent work may be performed on the patient.

16. The method of claim 10, wherein the procedure is associated with dilating stenosis or repairing an area of the patient.

17. A system, comprising:
an introducer, which includes:
a first lumen operable to be used in a procedure involving a vessel of a patient; and
a second lumen operable to be used in conjunction with the first lumen in the procedure, wherein the first lumen is operable to receive a first wire, which can be inserted in the first lumen and positioned in a first direction, and wherein the second lumen is operable to receive a second wire, which can be inserted in the second lumen and positioned in an opposite direction, which is not the same as the first direction.

18. The system of claim 17, further comprising:
a dilator operable to dilate the vessel in conjunction with the procedure.

19. The system of claim 17, further comprising:
a sheath, which is operable to deliver the first wire to the vessel.

20. The system of claim 17, wherein the second lumen includes a port that is operable to perform a bleed off function for the procedure.
